(19) United States
(12) Patent Application Publication
(10) Pub. No.: US 2024/0238515 A1
(43) Pub. Date: Jul. 18, 2024

(54) Vital Sign Monitoring And Naloxone Delivery Device

(71) Applicant: James Sinclair, Los Angeles, CA (US)

(72) Inventor: James Sinclair, Los Angeles, CA (US)

(21) Appl. No.: 18/098,545

(22) Filed: Jan. 18, 2023

(51) Int. Cl. A61M 5/172; A61M 5/142

(52) U.S. Cl. CPC A61M 5/1723; A61M 5/14244; A61M 2205/0216; A61M 2205/18; A61M 2205/3553; A61M 2205/502; A61M 2205/505; A61M 2230/20

(57) ABSTRACT

A vital sign monitoring and naloxone delivery device for reducing adverse outcomes of opioid overdoses includes a housing, which contains a battery, a pump, and a microprocessor, and to which band is attached. The band attaches the housing to an arm of a user. A pulse oximeter and an injector, which are attached to a bottom of the housing, the battery, and the pump are operationally engaged to the microprocessor. The injector is operationally engaged to the pump. The pulse oximeter measures a blood oxygenation level and a pulse rate of the user and relays the measurements to the microprocessor, which then determines an opioid overdose. A cartridge containing naloxone is inserted through a cutout in the housing to engage the cartridge to the pump. Upon determination of the overdose, the microprocessor actuates the pump and the injector to inject the user with naloxone to counter the effects an opioid.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to drug delivery devices and more particularly pertains to a new drug delivery device for reducing adverse outcomes of opioid overdoses. The present invention discloses a drug delivery device that reduces adverse outcomes of opioid overdoses, particularly amongst solitary opioid users.

### (2) Description of Related Art Including Information Disclosed Under 37 CFR 1.97 and 1.98

The prior art relates to drug delivery devices. The prior art includes devices that detect overdose events and automatically inject naloxone. The prior art also includes devices that detect overdose events and automatically send overdose alerts and locations of users to emergency services. What is lacking in the prior art is a drug delivery device that detects an overdose event and which can automatically inject up to two doses of naloxone, depending upon how a user responds, and which automatically sends an overdose alert and a location of the user to emergency services.

### BRIEF SUMMARY OF THE INVENTION

An embodiment of the disclosure meets the needs presented above by generally comprising a band that is attached to a housing, with the latter defining an interior space. The band is configured to selectively attach the housing to an arm of a user. A battery, a pump, and a microprocessor are attached to the housing and are positioned in the interior space. A pulse oximeter and an injector, which are attached to a bottom of the housing, the battery, and the pump are operationally engaged to the microprocessor. The injector also is operationally engaged to the pump. The pulse oximeter is in contact with skin of the arm upon attachment of the housing to the arm of the user and is configured to measure a blood oxygenation level and a pulse rate of the user and to relay the measurements to the microprocessor, enabling the microprocessor to determine an opioid overdose. A cutout positioned in the housing is configured for selective insertion of a cartridge that contains naloxone, thereby operationally engaging the cartridge to the pump. Upon determination of the overdose, the microprocessor is enabled to selectively actuate the pump and the injector to inject the user with naloxone to counter the effects an opioid.

Another embodiment of the disclosure includes a vital sign monitoring and naloxone delivery system, which comprises a vital sign monitoring and naloxone delivery device, as described in the disclosure above, and a cartridge containing naloxone that has been positioned through a cutout in the housing so that the cartridge is operationally engaged to the pump.

There has thus been outlined, rather broadly, the more important features of the disclosure in order that the detailed description thereof that follows may be better understood, and in order that the present contribution to the art may be better appreciated.

There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a vital sign monitoring and naloxone delivery device according to an embodiment of the disclosure.

FIG. 2 is a top view of an embodiment of the disclosure.

FIG. 3 is a side view of an embodiment of the disclosure.

FIG. 4 is a bottom view of an embodiment of the disclosure.

FIG. 5 is an in-use view of an embodiment of the disclosure.

FIG. 6 is a block diagram an embodiment of the disclosure.

### DETAILED DESCRIPTION OF THE INVENTION

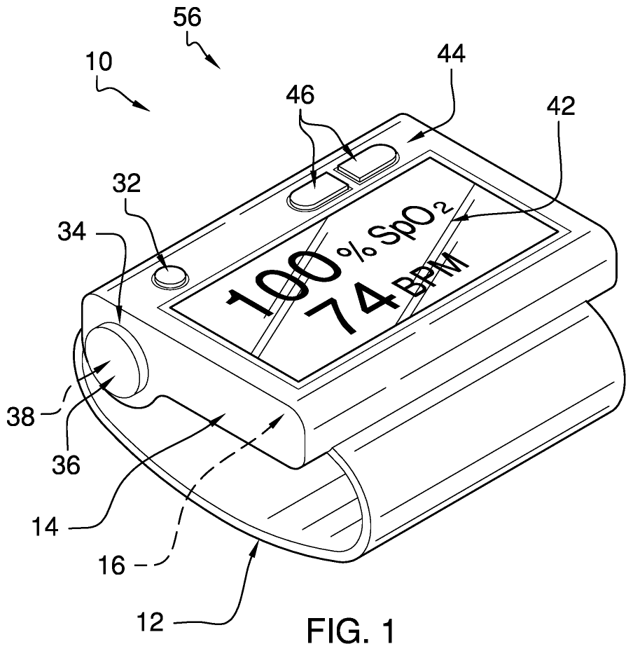

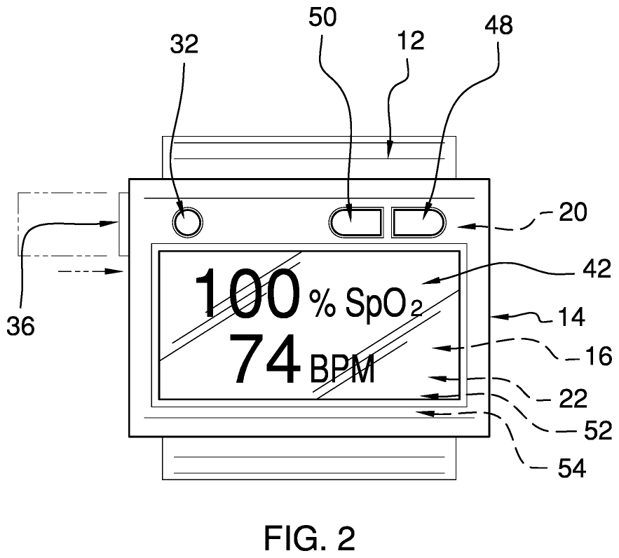

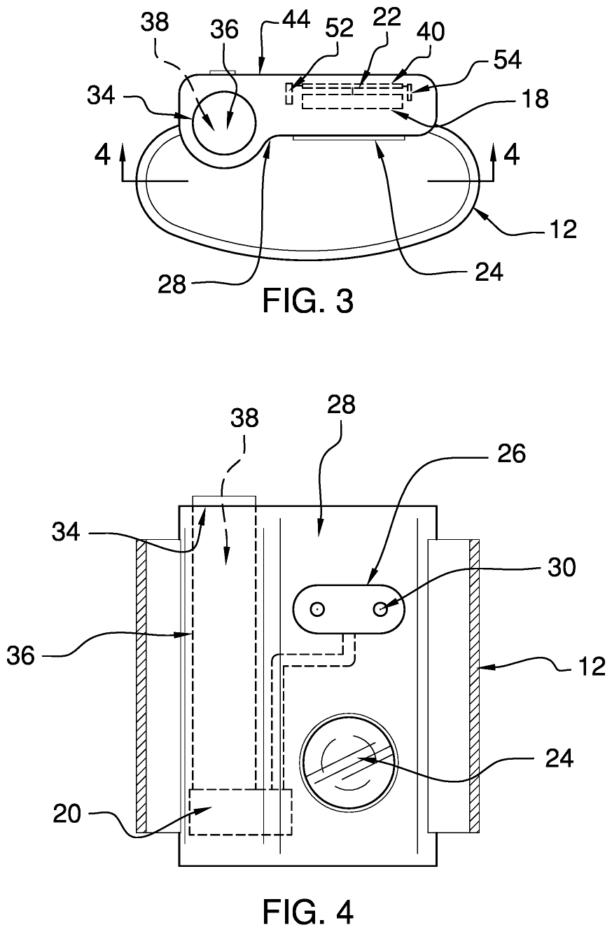

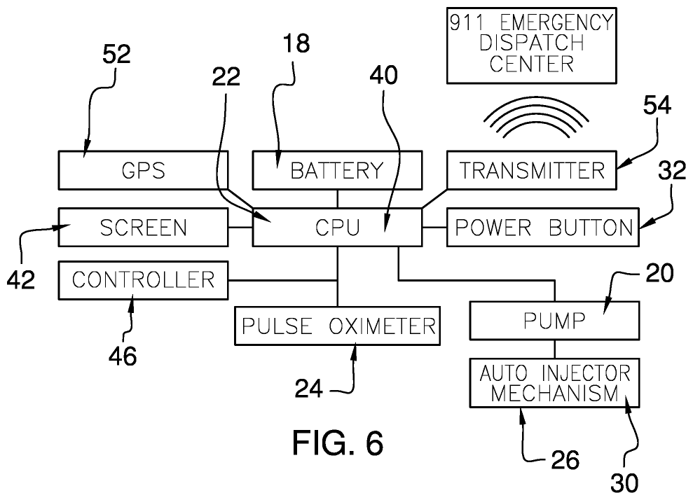

With reference now to the drawings, and in particular to FIGS. 1 through 6 thereof, a new drug delivery device embodying the principles and concepts of an embodiment of the disclosure and generally designated by the reference numeral 10 will be described.

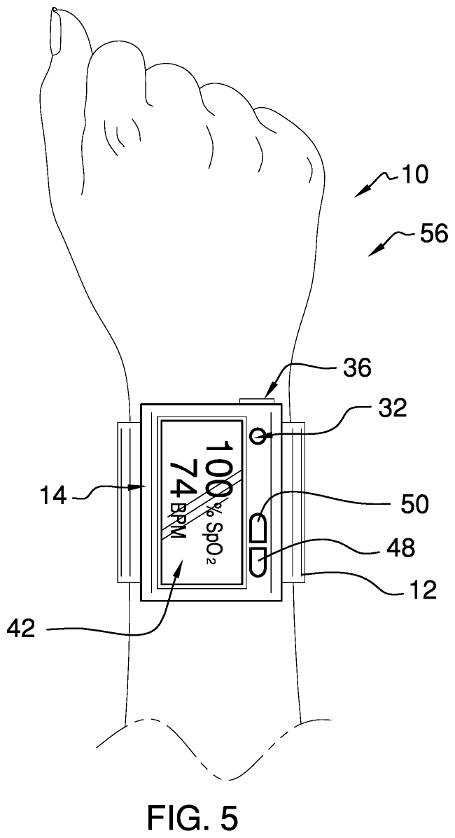

As best illustrated in FIGS. 1 through 6, the vital sign monitoring and naloxone delivery device 10 generally comprises a band 12 that is attached to a housing 14, with the latter defining an interior space 16. The band 12 is configured to selectively attach the housing 14 to an arm of a user, as shown in FIG. 5, wherein the housing is positioned around a wrist of the user. The band 12, which comprises elastomer, rubber, silicone, or the like, is resiliently stretchable. The band 12 is configured to be stretched for insertion of the arm of the user and to rebound to secure the housing 14 to the arm of the user. Other configurations of the band 12 are anticipated by the present invention.

For example, the band 12 may comprise a pair of complementary hook and loop straps, an elastomeric sleeve, or the like.

A battery 18, a pump 20, and a microprocessor 22 are attached to the housing 14 and are positioned in the interior space 16. A pulse oximeter 24 and an injector 26, which are attached to a bottom 28 of the housing 14, the battery 18, and the pump 20 are operationally engaged to the microprocessor 22. The injector 26 also is operationally engaged to the pump 20. The injector 26 comprises at least one needle 30, or other injection means, such as jet autoinjectors, or the like. The present invention also anticipates the injector 26 being removably attached to the housing 14 so that it can be replaced after use.

A power button 32 is attached to the housing 14 and is operationally engaged to the battery 18 and the microprocessor 22. The power button 32 is configured to be depressed a first time to operationally engage the microprocessor 22 to the battery 18 and to be depressed a second time to disengage the microprocessor 22 from the battery 18.

The pulse oximeter 24, which is in contact with skin of the arm upon attachment of the housing 14 to the arm of the user, is configured to measure a blood oxygenation level and a pulse rate of the user and to relay the measurements to the microprocessor 22, enabling the microprocessor 22 to determine an opioid overdose. A cutout 34 is positioned in the housing 14 and is configured for selective insertion of a cartridge 36 that contains naloxone 38, nalmefene, or the like, thereby operationally engaging the cartridge 36 to the pump 20. The cutout 34 is sized for insertion of a cartridge 36 that contains two doses of naloxone 38.

Upon determination of the overdose, the microprocessor 22 is enabled to selectively actuate the pump 20 and the injector 26 to inject the user with naloxone 38 to counter the effects an opioid. Programming code 40, which is positioned on the microprocessor 22, enables the microprocessor 22, at a predetermined interval after a first dose of the naloxone 38, to compare the measurements of the blood oxygenation level and the pulse rate to predetermined levels and to selectively actuate the pump 20 and the injector 26 to inject the user with a second dose of the naloxone 38, should the measurements be lower than the predetermined levels. The vital sign monitoring and naloxone delivery device 10 is anticipated to reduce adverse outcomes of opioid overdoses, particularly amongst solitary opioid users, but also for first time opioid users who do not have any knowledge of their opioid tolerance levels.

A display panel 42 is attached to a top 44 of the housing 14 and is operationally engaged to the microprocessor 22. The microprocessor 22 is enabled to selectively actuate the display panel 42 to present the measurements of the blood oxygenation level and the pulse rate. The display panel 42 allows the user to self-monitor their measurements to assess whether or not they are heading toward an overdose event.

A controller 46 is attached to the housing 14 and is operationally engaged to the microprocessor 22. The controller 46 is configured to be manipulated by a user to control a brightness of the display panel 42. The controller 46 may comprise an increase button 48 and a decrease button 50, or other controlling means, such as dials, slides, or the like. The increase button 48 and the decrease button 50 are configured for selective depression to increase and to decrease the brightness of the display panel 42, respectively.

A receiver 52 and a transmitter 54 are attached to the housing 14, are positioned in the interior space 16, and are operationally engaged to the microprocessor 22. The receiver 52 is Global Positioning System enabled and thus is configured to receive positional coordinates of the user. The microprocessor 22 is enabled to selectively actuate the transmitter 54 to transmit an overdose alert and the positional coordinates of the user. The transmitter 54 can transmit the overdose alert and the positional coordinates of the user to emergency services, thereby facilitating a rapid response time to address the overdose event and to minimize adverse outcomes of the overdose event.

The present invention also includes a vital sign monitoring and naloxone delivery system 56, which comprises a vital sign monitoring and naloxone delivery device 10, as described in the specification above, and a cartridge 36 containing naloxone 38 that has been positioned through a cutout 34 in the housing 14 so that the cartridge 36 is operationally engaged to the pump 20. The vital sign monitoring and naloxone delivery system 56 also may include programming code 40, per the specification above. The programming code 40 enables the microprocessor 22, at a predetermined interval after a first dose of the naloxone 38, to compare the measurements of the blood oxygenation level and the pulse rate to predetermined levels and to selectively actuate the pump 20 and the injector 26 to inject the user with a second dose of the naloxone 38, should the measurements be lower than the predetermined levels.

The vital sign monitoring and naloxone delivery system 56 also may include a receiver 52 and a transmitter 54, per the specification above. The transmitter 54 can transmit the overdose alert and the positional coordinates of the user to emergency services, thereby facilitating a rapid response time to address the overdose event and to minimize adverse outcomes of the overdose event.

In use, prior to injecting or otherwise using an opioid, a user inserts a cartridge 36 containing naloxone 38 through the cutout 34 in the housing 14 to operationally engage the cartridge 36 to the pump 20. The user inserts their arm through the band 12 so that the housing 14 is attached to their arm with the pulse oximeter 24 in contact with the skin of the arm. The use then pushes the power button 32 and confirms that the pulse oximeter 24 is operating by looking at the display panel 42. Should the microprocessor 22 subsequently determine an opioid overdose, it actuates the pump 20 and the injector 26 to inject the user with naloxone 38. The programming code 40 enables the microprocessor 22 to compare the measurements of the blood oxygenation level and the pulse rate to predetermined levels and to selectively actuate the pump 20 and the injector 26 to inject the user with a second dose of the naloxone 38, should the measurements be lower than the predetermined levels.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A vital sign monitoring and naloxone delivery device comprising:
a housing defining an interior space;
a band attached to the housing and being configured for selectively attaching the housing to an arm of a user;
a battery attached to the housing and positioned in the interior space;
a microprocessor attached to the housing, positioned in the interior space, and operationally engaged to the battery;
a pulse oximeter attached to a bottom of the housing and operationally engaged to the microprocessor, such that the pulse oximeter is in contact with skin of the arm upon attachment of the housing to the arm of the user, wherein the pulse oximeter is configured for measuring a blood oxygenation level and a pulse rate of the user and for relaying the measurements to the microprocessor, enabling the microprocessor for determining an opioid overdose;
a pump attached to the housing, positioned in the interior space, and operationally engaged to the microprocessor;
a cutout positioned in the housing, wherein the cutout is configured for selective insertion of a cartridge containing naloxone, such that the cartridge is operationally engaged to the pump; and
an injector attached to the bottom of the housing and operationally engaged to the pump and the microprocessor, wherein the microprocessor is enabled for selectively actuating the pump and the injector upon determination of the overdose for injecting the user with naloxone to counter the effects an opioid.

2. The vital sign monitoring and naloxone delivery device of claim 1, wherein the band is resiliently stretchable, wherein the band is configured for stretching for insertion of the arm of the user and for rebounding for securing the housing to the arm of the user.

3. The vital sign monitoring and naloxone delivery device of claim 2, wherein the band comprises elastomer, rubber, or silicone.

4. The vital sign monitoring and naloxone delivery device of claim 1, further including a power button attached to the housing and operationally engaged to the battery and the microprocessor, wherein the power button is configured for depressing a first time for operationally engaging the microprocessor to the battery and for depressing a second time for disengaging the microprocessor from the battery.

5. The vital sign monitoring and naloxone delivery device of claim 1, wherein the cutout is sized for insertion of a cartridge containing two doses of naloxone.

6. The vital sign monitoring and naloxone delivery device of claim 1, further including programming code positioned on the microprocessor enabling the microprocessor, at a predetermined interval after a first dose of the naloxone, for comparing the measurements of the blood oxygenation level and the pulse rate to predetermined levels and for selectively actuating the pump and the injector for injecting the user with a second dose of the naloxone should the measurements be lower than the predetermined levels.

7. The vital sign monitoring and naloxone delivery device of claim 1, wherein the injector comprises at least one needle.

8. The vital sign monitoring and naloxone delivery device of claim 1, further including a display panel attached to a top of the housing and operationally engaged to the microprocessor, such that the microprocessor is enabled for selectively actuating the display panel for presenting the measurements of the blood oxygenation level and the pulse rate.

9. The vital sign monitoring and naloxone delivery device of claim 8, further including a controller attached to the housing and operationally engaged to the microprocessor, wherein the controller is configured for being manipulated by a user for controlling a brightness of the display panel.

10. The vital sign monitoring and naloxone delivery device of claim 9, wherein the controller comprises an increase button and a decrease button, wherein the increase button and the decrease button are configured for selective depression for increasing and decreasing the brightness of the display panel, respectively.

11. The vital sign monitoring and naloxone delivery device of claim 1, further including:
a receiver attached to the housing, positioned in the interior space, and operationally engaged to the microprocessor, the receiver being Global Positioning System enabled, wherein the receiver is configured for receiving positional coordinates of the user; and
a transmitter attached to the housing, positioned in the interior space, and operationally engaged to the microprocessor, wherein the microprocessor is enabled for selectively actuating the transmitter for transmitting an overdose alert and the positional coordinates of the user.

12. A vital sign monitoring and naloxone delivery system comprising:
a housing defining an interior space;
a band attached to the housing and being configured for selectively attaching the housing to an arm of a user;
a battery attached to the housing and positioned in the interior space;
a microprocessor attached to the housing, positioned in the interior space, and operationally engaged to the battery;
a pulse oximeter attached to a bottom of the housing and operationally engaged to the microprocessor, such that the pulse oximeter is in contact with skin of the arm upon attachment of the housing to the arm of the user, wherein the pulse oximeter is configured for measuring a blood oxygenation level and a pulse rate of the user and for relaying the measurements to the microprocessor, enabling the microprocessor for determining an opioid overdose;
a pump attached to the housing, positioned in the interior space, and operationally engaged to the microprocessor;
a cartridge containing naloxone positioned through a cutout in the housing, such that the cartridge is operationally engaged to the pump; and
an injector attached to the bottom of the housing and operationally engaged to the pump and the microprocessor, wherein the microprocessor is enabled for selectively actuating the pump and the injector upon determination of the overdose for injecting the user with naloxone to counter the effects an opioid.

13. The vital sign monitoring and naloxone delivery system of claim 12, further including programming code positioned on the microprocessor enabling the microprocessor, at a predetermined interval after a first dose of the naloxone, for comparing the measurements of the blood oxygenation level and the pulse rate to predetermined levels and for selectively actuating the pump and the injector for injecting the user with a second dose of the naloxone should the measurements be lower than the predetermined levels.

14. The vital sign monitoring and naloxone delivery system of claim 12, further including:
a receiver attached to the housing, positioned in the interior space, and operationally engaged to the microprocessor, the receiver being Global Positioning System enabled, wherein the receiver is configured for receiving positional coordinates of the user; and
a transmitter attached to the housing, positioned in the interior space, and operationally engaged to the microprocessor, wherein the microprocessor is enabled for selectively actuating the transmitter for transmitting an overdose alert and the positional coordinates of the user.

15. A vital sign monitoring and naloxone delivery device comprising:
a housing defining an interior space;
a band attached to the housing and being configured for selectively attaching the housing to an arm of a user, the band being resiliently stretchable, wherein the band is configured for stretching for insertion of the arm of the user and for rebounding for securing the housing to the arm of the user, the band comprising elastomer, rubber, or silicone;
a battery attached to the housing and positioned in the interior space;
a microprocessor attached to the housing, positioned in the interior space, and operationally engaged to the battery;
a power button attached to the housing and operationally engaged to the battery and the microprocessor, wherein the power button is configured for depressing a first time for operationally engaging the microprocessor to the battery and for depressing a second time for disengaging the microprocessor from the battery;
a pulse oximeter attached to a bottom of the housing and operationally engaged to the microprocessor, such that the pulse oximeter is in contact with skin of the arm upon attachment of the housing to the arm of the user, wherein the pulse oximeter is configured for measuring a blood oxygenation level and a pulse rate of the user and for relaying the measurements to the microprocessor, enabling the microprocessor for determining an opioid overdose;
a pump attached to the housing, positioned in the interior space, and operationally engaged to the microprocessor;
a cutout positioned in the housing, wherein the cutout is configured for selective insertion of a cartridge containing naloxone, such that the cartridge is operationally engaged to the pump, the cutout being sized for insertion of a cartridge containing two doses of naloxone;
an injector attached to the bottom of the housing and operationally engaged to the pump and the microprocessor, wherein the microprocessor is enabled for selectively actuating the pump and the injector upon determination of the overdose for injecting the user with naloxone to counter the effects an opioid, the injector comprising at least one needle;
programming code positioned on the microprocessor enabling the microprocessor, at a predetermined interval after a first dose of the naloxone, for comparing the measurements of the blood oxygenation level and the pulse rate to predetermined levels and for selectively actuating the pump and the injector for injecting the user with a second dose of the naloxone should the measurements be lower than the predetermined levels;
a display panel attached to a top of the housing and operationally engaged to the microprocessor, such that the microprocessor is enabled for selectively actuating the display panel for presenting the measurements of the blood oxygenation level and the pulse rate;
a controller attached to the housing and operationally engaged to the microprocessor, wherein the controller is configured for being manipulated by a user for controlling a brightness of the display panel, the controller comprising an increase button and a decrease button, wherein the increase button and the decrease button are configured for selective depression for increasing and decreasing the brightness of the display panel, respectively;
a receiver attached to the housing, positioned in the interior space, and operationally engaged to the microprocessor, the receiver being Global Positioning System enabled, wherein the receiver is configured for receiving positional coordinates of the user; and
a transmitter attached to the housing, positioned in the interior space, and operationally engaged to the microprocessor, wherein the microprocessor is enabled for selectively actuating the transmitter for transmitting an overdose alert and the positional coordinates of the user.
